(19) United States
(12) Patent Application Publication
(10) Pub. No.: US 2007/0093699 A1
(43) Pub. Date: Apr. 26, 2007

(54) Magnification ratio adjustment for observing blood flow in capillary vessels

(76) Inventor: Teruo Takeno, Kyoto (JP)

Correspondence Address: MERCHANT & GOULD PC, P.O. BOX 2903, MINNEAPOLIS, MN, 55402-0903, US

(21) Appl. No.: 11/522,094

(22) Filed: Sep. 14, 2006

(30) Foreign Application Priority Data
Mar. 16, 2004 (JP) JP2004-261694

(51) Int. Cl. A61B 5/00

(52) U.S. Cl. 600/316

(57) ABSTRACT

Blood flow in capillary vessels can be observed by using a magnification ratio that can be altered by the magnification ratio adjustor without causing blurring so that zooming operations can be easily performed. An objective lens can easily be centered relative to a fingertip, for example, without causing a burden to patients by having the patients themselves move their fingertips to bring a desired image into focus. Heat dissipation from the illuminator can be facilitated, and the imaging units can be more compactly designed.

### RELATED APPLICATIONS

This application claims the benefit under 35 USC §120 of a parent Patent Cooperation Treaty application JP2004-261694 filed Mar. 16, 2004, published as kokai 2005-261494.

### FIELD OF THE INVENTION

The present disclosure generally relates to observing blood flow in capillary vessels in, for example, the nail epithelium of a fingertip. More particularly, the present disclosure relates to magnification ratio adjustment.

### BACKGROUND

Health care providers can observe the blood flow in capillary vessels of a patient in order to assess the health of the patient. Accordingly, a magnification device can be used to observe the fine details of the capillary vessels. However, the location and size of capillary vessels in a patient's finger, for example, can vary depending on the particular patient being observed. Magnification devices can adjust the magnification ratio (“zoom”) but often lose their focus when zooming. The loss of focus provides a distraction for the health care provider when zooming and panning across the capillary vessels of a patient.

### SUMMARY OF THE INVENTION

The present disclosure provides exemplary embodiments of the invention, which is defined by the claims as recited herein. In various embodiments, magnification ratio adjustment for observing blood flow in capillary vessels can be practiced using an apparatus that comprises a light source for illuminating, for example, a fingertip with light, and an optics imaging and processing component for producing an enlarged image of the fingertip. The enlarged image allows blood flow in a capillary vessel at the fingertip to be observed by a health care provider, for example.

The optics imaging and processing component in the embodiment comprises a lens-barrel; a magnification ratio adjustor having an objective lens that is provided at the lower end of the lens-barrel (31) and an imaging component having an image sensor (such as a CCD, a CMOS sensor, and the like) and being provided at the upper end of the lens-barrel. The lens-barrel comprises an interval adjustment mechanism that is disposed between the imaging component and the lens-barrel in order to adjust the interval therebetween. The interval adjustment mechanism is arranged such that the focal point can be easily maintained when zooming using the interval adjustment mechanism.

In another embodiment, the interval that extends from the objective lens to the image sensor is set by adjusting the interval between the imaging component and the lens-barrel with the interval adjustment mechanism, so that a focus point of the light passing through the magnification ratio adjustor matches the position of the image sensor.

In yet another embodiment, the interval adjustment mechanism comprises a lower cylinder fixed to the lens-barrel and an upper cylinder fixed to the imaging component, where the upper cylinder is threadably mounted onto the outer or inner surface of the lower cylinder so that the interval can be set by rotating the upper cylinder in relation to the lower cylinder.

In yet another embodiment, the optics processing and imaging component is mounted to a vertical pole having a supporting arm which allows the component to rotate on a horizontal plane that is perpendicular to the vertical pole and to move up or down along the vertical pole. The supporting arm comprises an X-axis stage, which allows the optics processing and imaging component to move in a horizontal direction perpendicular to the vertical pole, a Y-axis stage, which allows the component to move in another horizontal direction perpendicular to the horizontal direction, and a Z-axis stage, which allows the component to move in the vertical direction.

In yet another embodiment, the illuminator comprises a light source provided in a housing having an opening for emitting the light from the light source to the outside of the housing through a condensing lens, and a fan for exhausting heat of the inside of the housing to the outside, the opening comprising an aperture mechanism of the light, and a mirror surface being provided on the opposite side of the opening.

In yet another embodiment, the illuminator is provided with a mounting arm on the same base as the vertical pole. The mounting arm comprises two ball joints and a 360 degree-rotatable axis provided between the ball joints so that the illuminator can be placed in any desired orientation.

### BRIEF DESCRIPTION OF THE DRAWINGS

Non-limiting and non-exhaustive embodiments are described with reference to the following drawings.

FIG. 1 is a perspective view that illustrates an apparatus for observing blood flow of capillary vessels.

FIG. 2 illustrates a top view of the apparatus.

FIG. 3 illustrates a side view from arrow III of FIG. 1.

FIG. 4 illustrates a side view from arrow IV of FIG. 1.

FIG. 5 illustrates a side view from arrow V of FIG. 1.

FIG. 6 illustrates a view of a cross section of an optics processing and imaging section.

FIG. 7 is an enlarged view of area A from FIG. 6.

FIG. 8 illustrates a side view of a supporting arm.

FIG. 9 illustrates an exploded perspective view of FIG. 8.

FIG. 10 illustrates a sectional view of an illuminator.

FIG. 11 is an illustration of a mounting arm.

FIG. 12 is an illustration of an enlarged image of capillary blood vessels.

### DETAILED DESCRIPTION

Various embodiments will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

Throughout the specification and claims, the following terms take at least the meanings explicitly associated herein, unless the context clearly dictates otherwise. The meanings identified below are not intended to limit the terms, but merely provide illustrative examples for use of the terms. The meaning of “a,” “an,” and “the” may include reference to both the singular and the plural. The meaning of “in” may include “in” and “on.” The term “coupled” can mean a direct connection between items, an indirect connection through one or more intermediaries, or communication between items in a manner that may not constitute a connection.

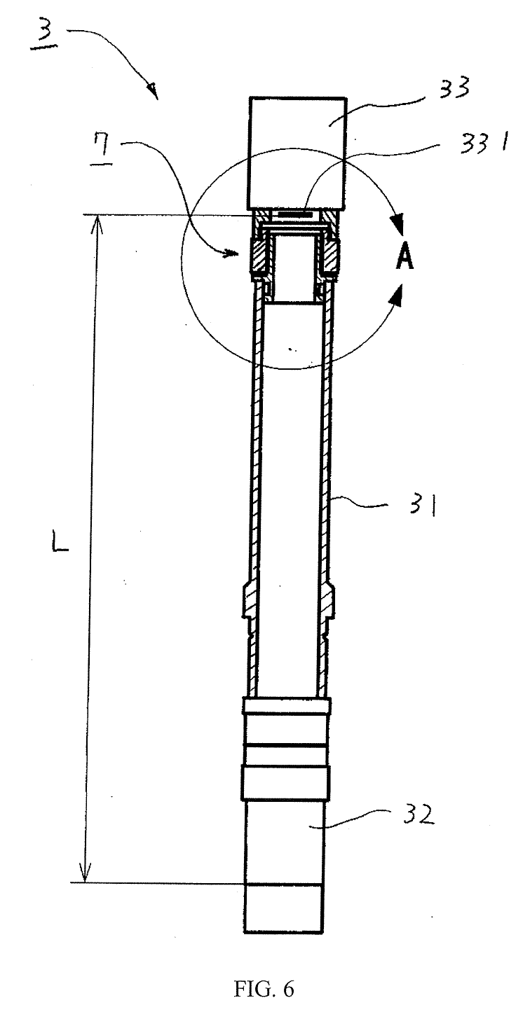

Briefly stated, the present disclosure generally relates to observing blood flow in a capillary vessel. An apparatus for observing blood flow in a capillary vessel is arranged having a first length that extends from an objective lens to an image sensor, so that a focus point of the light passing through the magnification ratio adjustor 922 is focused upon the image sensor. However, because it is difficult, in practice, to assemble the optics processing and imaging component with sufficiently strict tolerances, an error may occur with respect to a first length “L” (see FIG. 6, for example) that extends from the objective lens to the image sensor.

Accordingly, when a user changes a magnification ratio with the magnification ratio adjustor (such as when zooming in or out), a shift of a focus point may result which causes the desired image to be out of focus. Conventional methods often include an additional focusing step which is conducted by moving of an optics processing and imaging component in an up or down direction. However, this operation provides a distraction and requires extra care for dealing with the tedious operation of maintaining focus.

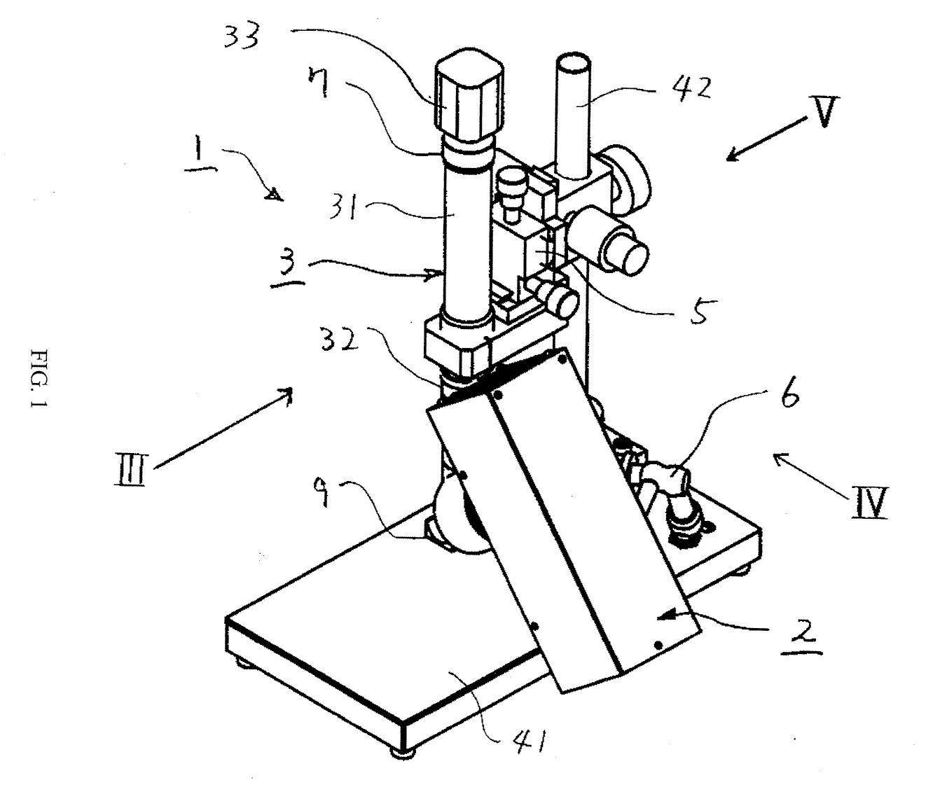

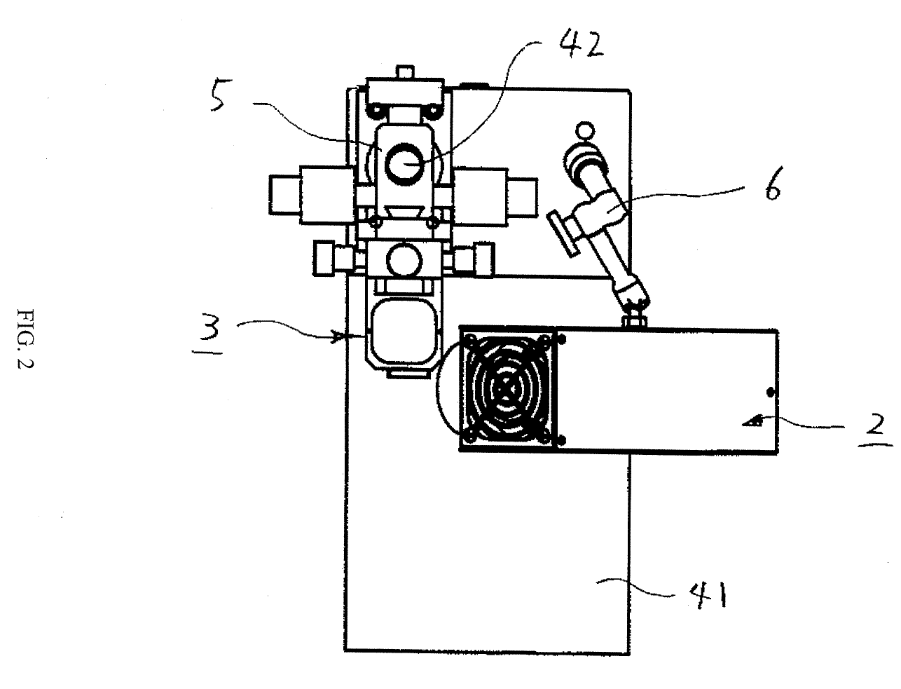

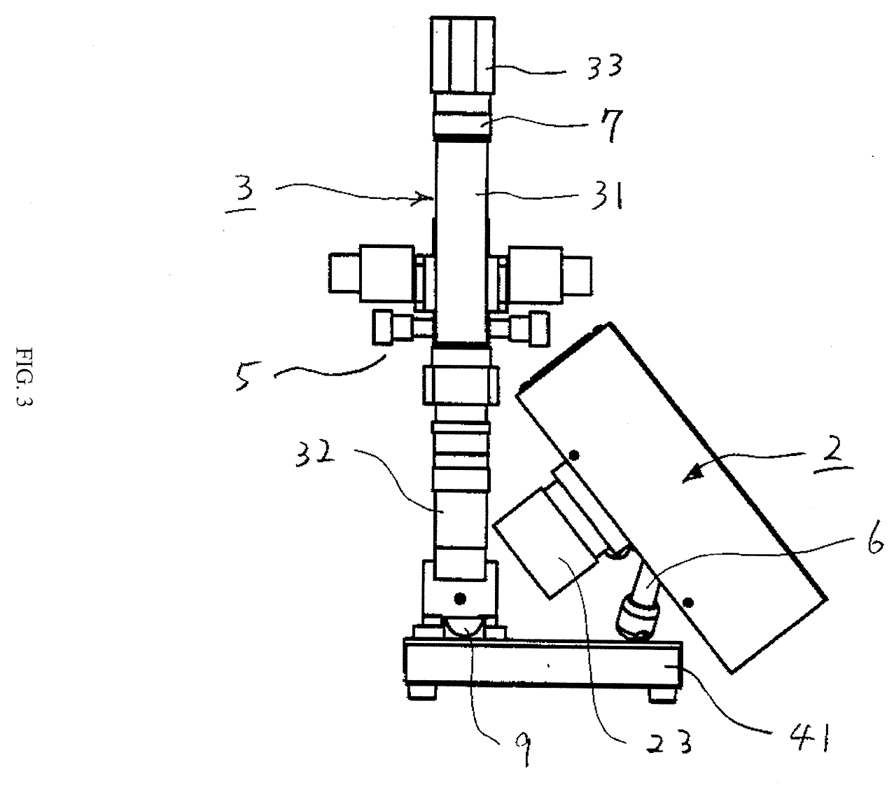

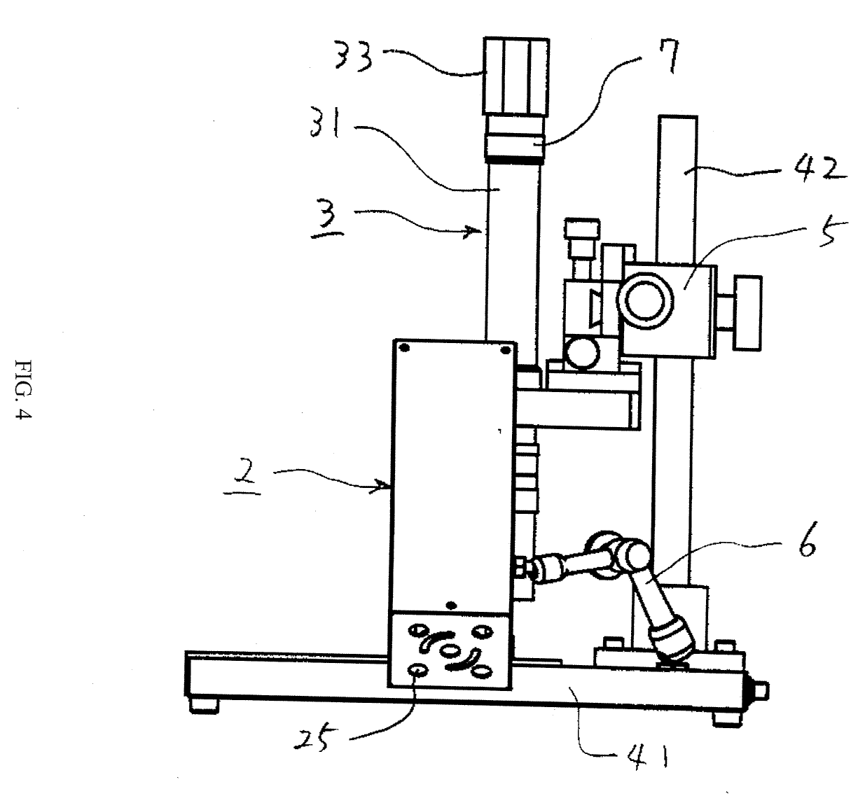

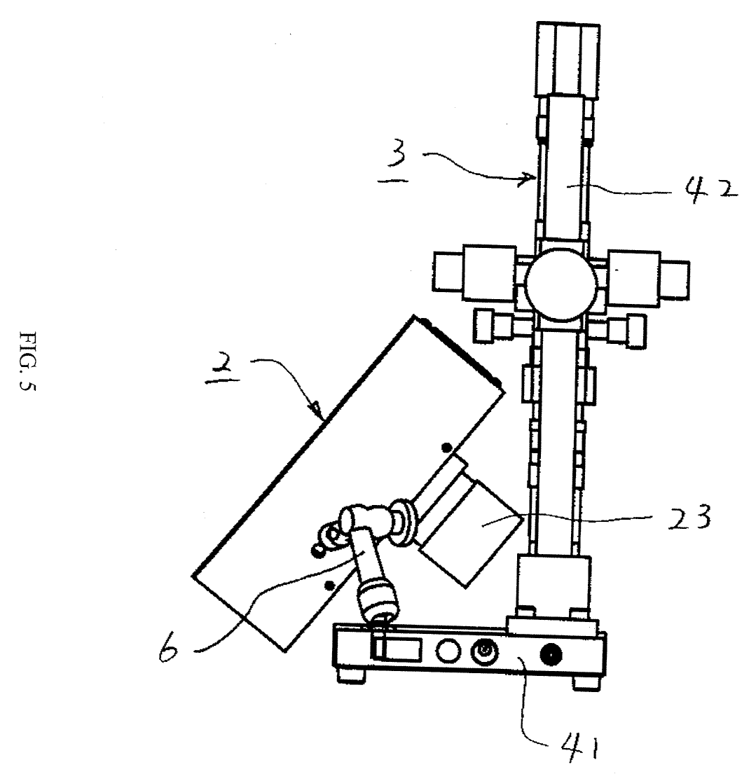

FIG. 1 is a perspective view that illustrates an apparatus for observing blood flow of capillary vessels. FIG. 2 illustrates a top view of the apparatus. FIG. 3 illustrates a side view from arrow III of FIG. 1. FIG. 4 illustrates a side view from arrow IV of FIG. 1. FIG. 5 illustrates a side view from arrow V of FIG. 1.

In an embodiment, an apparatus 1 comprises a light source 2 for illuminating a fingertip with light, and an optics imaging and processing component 3 for producing an enlarged image of the fingertip. The enlarged image allows blood flow in a capillary vessel at the fingertip to be observed by a health care provider, for example, viewing a display (not shown).

The optics imaging and processing component 3 in the embodiment comprises a lens-barrel; a magnification ratio adjustor having an objective lens that is provided at the lower end of the lens-barrel 31 and an imaging component having an image sensor (such as a CCD, a CMOS sensor, and the like) and being provided at the upper end of the lens-barrel. The lens-barrel 31 comprises an interval adjustment mechanism 7 that is disposed between the imaging component and the lens-barrel in order to adjust the interval therebetween. The interval adjustment mechanism 7 is arranged such that the focus point can be easily maintained when zooming using the interval adjustment mechanism.

Optical processing/imaging component 3 is mounted to vertical pole 42 with supporting arm 5, the vertical pole 42 being provided on base 41 in a vertical direction. Illuminator 2 is mounted on the base 41 with mounting arm 6.

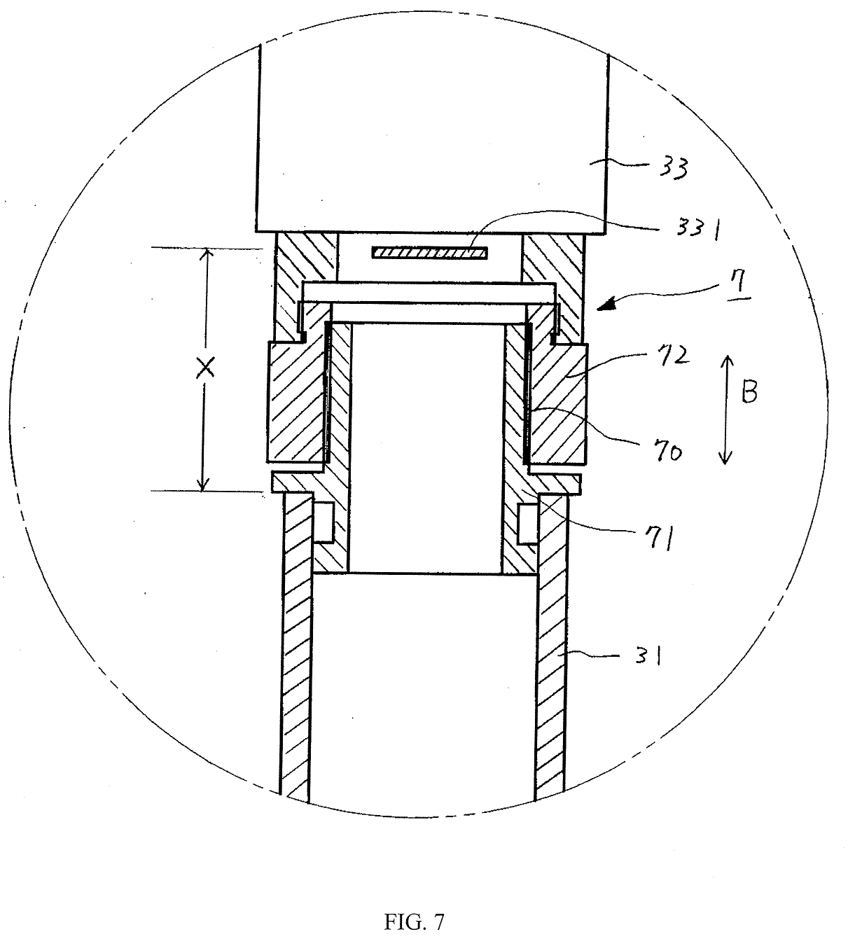

The optics processing and imaging component 3, as shown in FIG. 6 (which is a partly-sectional view of the whole part of the component), is configured to have a lens-barrel with a cavity of the inside, magnification ratio adjustor 32 with a objective lens (not shown in figures), and imaging component 33 with CCD 331 (as discussed above other imaging sensors can be used). The example apparatus in various embodiments further comprises interval adjustment mechanism 7 in optical processing/imaging component 3. As shown in FIGS. 6 and 7 (which is an enlarged view of part A from FIG. 6), the interval adjustment mechanism 7 is provided between lens-barrel 31 and imaging component 33 so that the interval X can be adjusted.

Interval adjustment mechanism 7 comprises lower cylinder 71 that is attached to lens-barrel 31, and upper cylinder 72 that is attached to imaging component 33. Upper cylinder 72 is threadably mounted onto the outer surface of lower cylinder 71 (for example, through a spiral screw mechanism) so that the upper cylinder can move in a longitudinal direction (as shown by arrow B) as it rotates in relation to lower cylinder 71. In other words, interval adjustment mechanism 7 can be configured to alter interval X by rotating upper cylinder 72. Section 70 is helically driven. Both cylinders 71 and 72 are designed not to obstruct light passing from magnification ratio adjustor 32 to a sensor, such as CCD 331.

In the embodiment, a first interval L from the object lens of magnification ratio adjustment 32 to CCD 331 is adjustable to match with a reference interval by adjusting interval X by using interval adjustment mechanism 7. The term “reference interval” references the interval that extends from an objective lens to a sensor, which is determined in its design so that a focus point of the light passing through magnification ratio adjustor 32 is focused upon the sensor. In many cases, the apparatus 1 has an error in dimension L when assembled (due to imprecise tolerances, for example). The manufacturing error can be corrected by adjusting interval X by using interval adjustment mechanism 7.

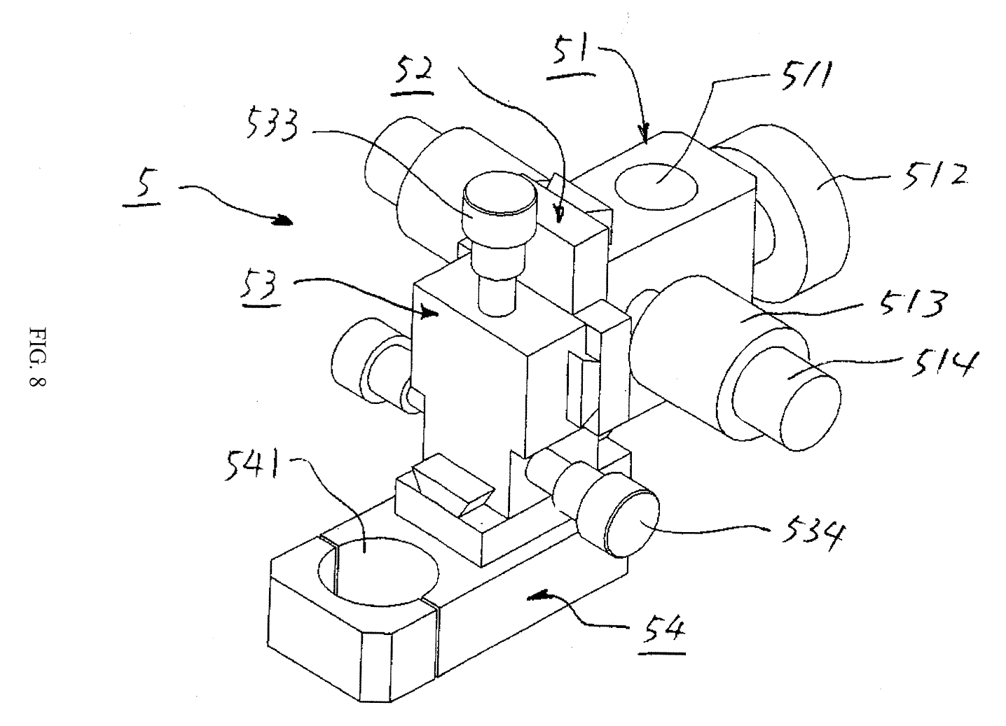

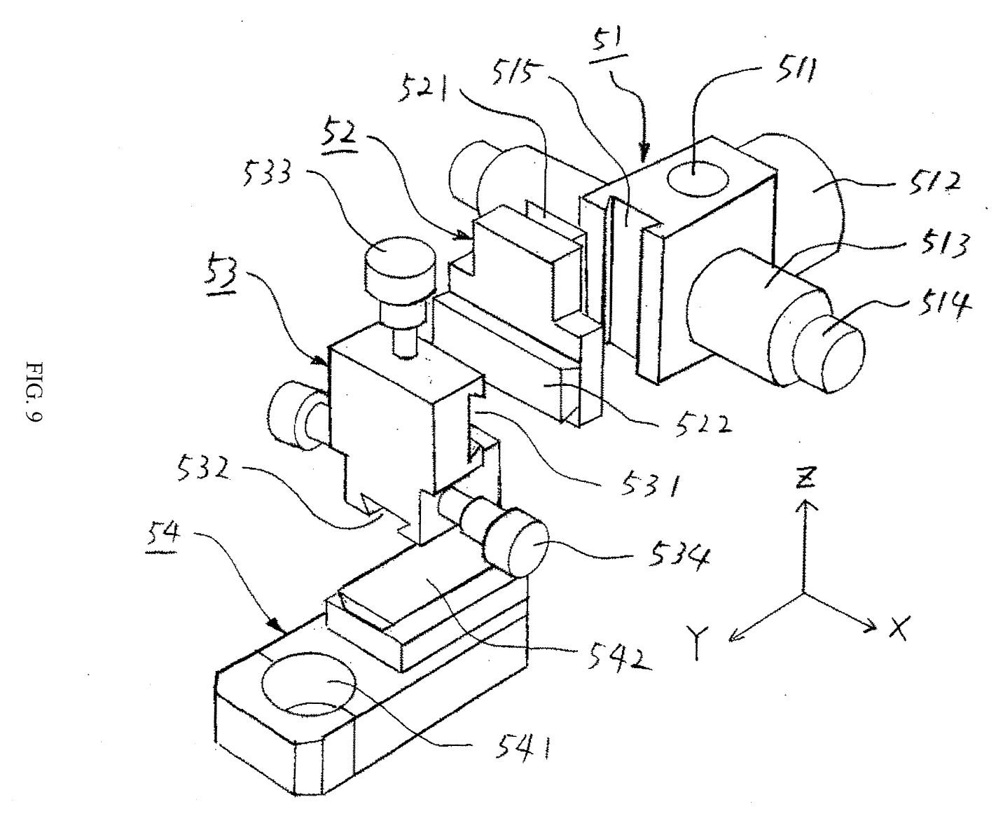

FIG. 8 illustrates a side view of supporting arm 5. FIG. 9 illustrates an exploded perspective view of FIG. 8. Arrows in FIG. 9 indicate a Y-axis direction, an X-axis direction, and a Z-axis direction. The X-axis is one of the horizontal directions, the Y-axis is another horizontal direction that is perpendicular to the X-axis direction (and that defines a horizontal plane comprising the X and Y axes), and Z-axis is the vertical direction. Supporting arm 5 includes a first stage 51, a second stage 52, a third stage 53, and a forth stage 54. Supporting arm 5 is mounted on vertical pole 42 with the first stage 51 and also supports optics processing and imaging component 3 with the forth stage 54.

The first stage 51 has an aperture 511 into which vertical pole 42 can be inserted. First stage 51 can be affixed to vertical pole 42 inserted through the penetration hole 511 by tightening first knob 512, and can also be rotatably mounted in a horizontal direction and moved up and down by loosening first knob 512. The first stage 51 also has a second knob 513 and a third knob 514. The first stage 51 further has a first recessed portion (or concave) 515 that generally extends in the Z-axis direction. The first recessed portion typically has a wider bottom than its opening.

The second stage 52 has a first convex portion 512 stretching in the Z-axis direction, the shape of which slideably fits that of the first recessed portion 515 of the first stage 51, and a second convex portion 522 extending in the X-axis direction. Both the convex portions 512 and 522 typically have wider top surfaces than the bottom surfaces.

The third stage 53 has a second recessed portion 531 stretching in the X-axis direction, and a third recessed portion 532 stretching in the Y-axis direction. Both recessed portions 531 and 532 have wider bottoms than their openings. The shape of the second recessed portion 531 slideably fits that of the second convex portion 522. The third stage 53 has a forth knob 533 and a fifth knob 534.

The forth stage 54 has an aperture 541 for holding the optics processing and imaging component 3. Stage 54 also has a third convex portion 542, the shape of which slideably fits that of the third recessed portion 532 of the third stage 53.

Accordingly, first, second, third and forth stages 51-54 can be coupled to supporting arm 5 as an assembly when the first convex portion 521 and the first recessed portion 515 are joined together, the second convex portion 522 and the second recessed portion 531 are joined together, and the third convex portion 542 and the third recessed portion 532 are joined together. The Z-axis stage can be coupled to the first convex portion 521 to allow rotation of an axis (not shown in figures) of the second and third knobs 513 and 514 as a rack and pinion mechanism. The second knob 513 can have a smaller number of pinions than that of the third knob 514 (those pinions are not shown in figures).

The X-axis stage can be provided with a linkage of the convex portion 522 to rotation of an axis (not shown) of the forth knob 533 as a rack and pinion mechanism. The Y-axis stage can be provided with a linkage of the third convex portion 542 to rotation of an axis (not shown) of the fifth knob 534 as a rack and pinion mechanism. In the example structures of the supporting arm 5, second stage 52 moves along the Z-axis direction in relation to the first stage 51 as the second knob 513 or the third knob 514 is rotated. The third stage 53 moves along the X-axis direction in relation to the second stage 52 as the forth knob is rotated, and the stage part 54 moves along the Y-axis direction in relation to the third stage 53 as the fifth knob 534 is rotated. With respect to adjustment using the second and third knobs 513 and 514, a rotation of the third knob 514 can provide a smaller amount of the movement in the Z-axis direction as compared to the amount of rotation provided by the second knob.

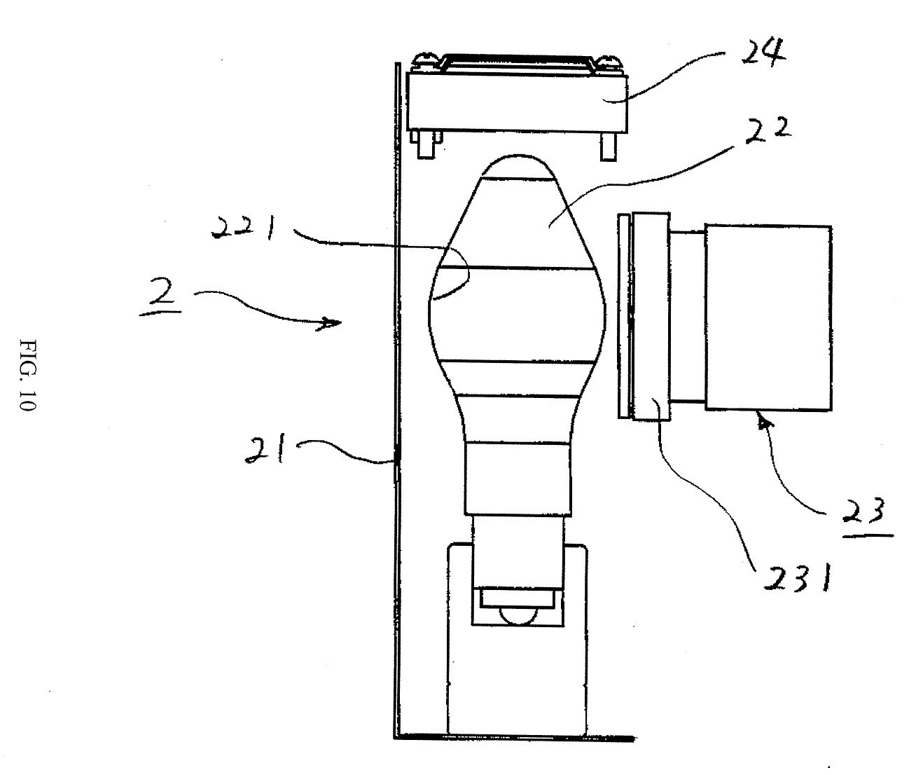

FIG. 10 illustrates a sectional view of illuminator 2. In an embodiment, illuminator 2 comprises a housing body 21, a light source 22 provided in the housing 21, an opening 23 for emitting the light from the light source 22 to the outside of the housing 21 through a condensing lens 231, and a fan 24 and air inlet 25 for exhausting heat of the inside of the housing 21 to the outside. The opening 23 typically has an aperture mechanism of the passing light (not shown). On the opposite side to the opening a coating of mirror surface is applied. The light source 22 can be, for example, a high pressure mercury lamp.

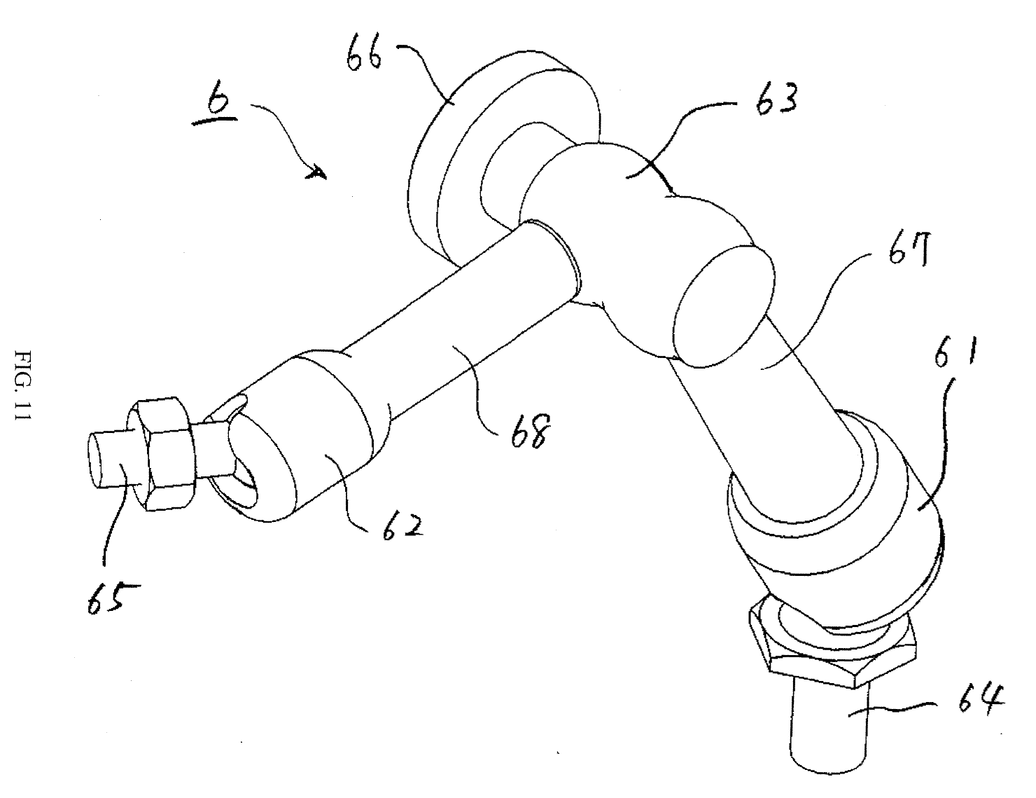

The illuminator 2 is mounted on the base 41 with a mounting arm 6 as shown in FIG. 11. The mounting arm has two ball joints 61, 62, and a 360 degree-rotatable axis 63 provided between the two ball joints. The ball joint 61 is also coupled with an edge 64 that can be fixed to the base 41. The ball joint 62 is also coupled with another edge 65 that can be fixed to the illuminator 2. A lock dial 66 can be provided on one end of the rotatable axis 63. The mounting arm 6 can be configured to lock any movements related to the joint balls 61, 62 the rotatable axis 63, and the parts of arm 67, 68 when the lock dial 66 is tightened.

In operation, apparatus 1 can be used as follows. The fingertip of a subject to be tested can be placed on a finger holder 9 as shown in FIG. 3. The illuminator is typically adjusted so that the fingertip on the holder 9 can be successfully exposed to the light (for example, by use of rotations at the joint ball 61, 62 and the axis 63). The illuminator can be fixed in a desired position by tightening of the lock dial 66. When the lock dial 66 is tightened, the first knob 512 can be loosened so the supporting arm 5 can be used to move the optics processing and imaging component 3 to where the component 3 is placed above the fingertip on the holder.

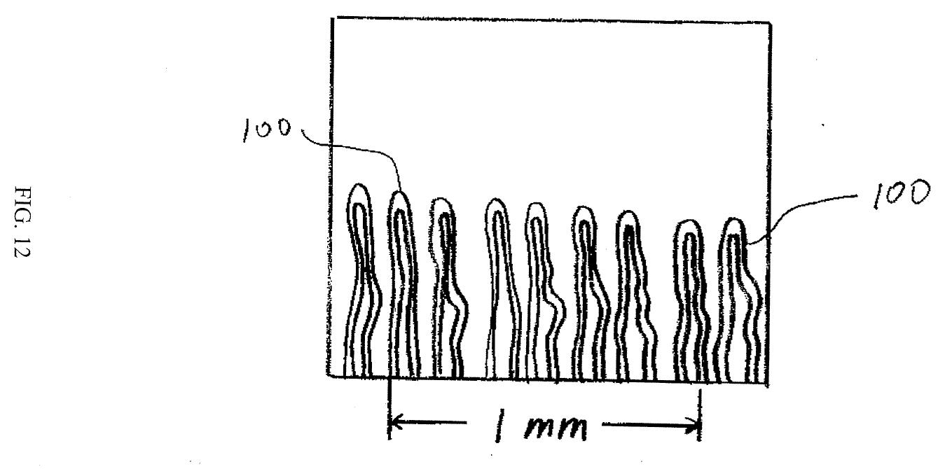

The fingertip on the holder 9 can be illuminated by turning on the light source 22 of the illuminator 2, while focusing the light from the source 22 through an aperture mechanism in the opening 23. A fan 24 can also be used to cool the illuminator 2. The objective lens of the magnification ratio adjustor 32 can be brought to a desired location adjacent to the fingertip on the holder 9 by sliding the second stage 52 with respect to the first stage 51 along the Z-axis direction by rotation of the second knob 513 or the third knob 514, and/or sliding of the third stage 53 with respect to the second stage 52 along the X-axis direction by rotation of the forth knob 533, and/or sliding of the forth stage 54 with respect to the third stage 53 along the Y-axis direction by rotation of the fifth knob 534. The magnification ratio (such as when zooming is) can be performed by using the magnification ratio adjustor 32. An image of the enlarged capillary vessels can be seen as illustrated by FIG. 12.

Although the invention has been described herein by way of exemplary embodiments, variations in the structures and methods described herein may be made without departing from the spirit and scope of the invention. For example, the positioning and/or sizing of the various components may be varied. Individual components and arrangements of components may be substituted as known to the art. Since many embodiments of the invention can be made without departing from the spirit and scope of the invention, the invention is not limited except as by the appended claims.

What is claimed is:

1. An apparatus for observing blood flow in a capillary vessel, comprising:
an illuminator for illuminating capillaries with light; and
an optics processing and imaging component for capturing an image of capillary blood flow, whereby blood flow in a capillary vessel can be observed in a displayed image, and wherein the optics processing and imaging component comprises:
a lens-barrel;
a magnification rate adjustor comprising an objective lens that is disposed at the lower portion of the lens-barrel;
an imaging component disposed at the upper portion of the lens-barrel; and
an interval adjustment mechanism disposed between the imaging sensor and the lens-barrel.

2. The apparatus of claim 1, wherein the distance from the objective lens to the imaging component is set by adjusting the interval between the imaging component and the lens-barrel by using the interval adjustment mechanism, whereby the light passing through the magnification ratio adjustor is focused on a sensor in the imaging component.

3. The apparatus of claim 1 wherein the interval adjustment mechanism comprises a lower cylinder fixed to the lens-barrel and a upper cylinder fixed to the imaging component, wherein the upper cylinder is threadably mounted onto the outer or inner surface of the lower cylinder so that the interval between the imaging component and the lens-barrel can be set by rotating the upper cylinder in relation to the lower cylinder.

4. The apparatus of claim 4, wherein the optics processing and imaging component is mounted to a vertical support member with a supporting arm that is configured to permit the rotation of the optics processing and imaging component in a horizontal plane perpendicular to the vertical support member and to move up and down along the vertical support member, wherein the supporting arm comprises an X-axis stage that allows the optics processing and imaging component to move in a first horizontal direction perpendicular to the vertical support member, a Y-axis stage that allows the component to move in a second horizontal direction perpendicular to the first horizontal direction, and a Z-axis stage that allows the component to move in a vertical direction.

5. The apparatus of claim 1, wherein the illuminator comprises a light source provided in a housing comprising an opening for emitting the light from the light source to the outside of the housing through a condensing lens wherein the opening comprises an aperture adjustment mechanism, the housing comprising a mirror surface being provided on the opposite side to the opening, and the housing comprising a fan for exhausting heat of the inside of the housing body to the outside.

6. The apparatus of claim 1, wherein the illuminator is provided with a mounting arm that is coupled to a same base to which the vertical support member is coupled, the mounting arm comprising, two ball joints rotatable 360 degrees about an axis between the ball joints whereby the illuminator can be placed in a desired position.

7. A method for observing blood flow in a capillary vessel, comprising:
illuminating capillaries with light for producing an image of the capillaries;
adjusting an interval between an objective lens that is disposed at the lower portion of a lens-barrel and an imaging component that is disposed at the upper portion of the lens-barrel, whereby the focus is maintained on the imaging component as the image is enlarged; and
capturing an image of capillary blood flow, whereby blood flow in a capillary vessel can be observed in a displayed image.

8. An apparatus for observing blood flow in a capillary vessel, comprising:
means for illuminating capillaries with light for producing an image of the capillaries;
means for adjusting an interval between an objective lens that is disposed at the lower portion of a lens-barrel and an imaging component that is disposed at the upper portion of the lens-barrel, whereby the focus is maintained on the imaging component as the image is enlarged; and
means for capturing an image of capillary blood flow, whereby blood flow in a capillary vessel can be observed in a displayed image.
